(19) United States
(12) Patent Application Publication
(10) Pub. No.: US 2005/0147974 A1
(43) Pub. Date: Jul. 7, 2005

(54) Luminescent, spherical, non-autofluorescent silica gel particles having variable emission intensities and frequencies

(76) Inventor: Detlef P. Muller-Shulte, Aachen (DE)

Correspondence Address: D Peter Hochberg Company, The Baker Building 6th Floor, 1940 East 6th Street, Cleveland, OH, 44114-2294, US

(21) Appl. No.: 10/509,712

(22) PCT Filed: Mar. 27, 2003
(86) PCT No.: PCT/EP03/03163

(30) Foreign Application Priority Data
Mar. 30, 2002 (DE) 102 14 019.7

(51) Int. Cl. C12Q 1/68; G01N 33/53; G02B 5/23

(52) U.S. Cl. 435/6; 435/7.1; 252/586

(57) ABSTRACT

Spherical, transparent silica gel particles with a high luminescence that are produced through the encapsulation of one or more different luminescent substances in a silica gel matrix. By varying the concentration of the luminescent substances and through the encapsulation of substances with different emission frequencies, the particles can be used as multiplex coding and marker systems in bioanalytics and diagnostics.

### CROSS REFERENCE TO RELATED APPLICATIONS

This application is a National Stage application of International Application No. PCT/EP03/03163, filed on Mar. 27, 2003, which claims priority of German Application No. 102 14 019.7, filed Mar. 30, 2002.

### FIELD OF THE INVENTION

The present invention relates to luminescent, spherical micro- and nanoparticles with changeable luminescence intensities, a process for their production and their use.

### DESCRIPTION OF THE PRIOR ART

Luminescence is defined for the following as the emission of light over the entire spectral range by substances that have been excited in advance through energy absorption. Fluorescence and phosphorescence also fall under this term.

Luminescent substances are in routine use today in the form of fluorescent or phosphorescent substances in the entire field of biochemical and medical analytics and diagnostics to identify analytes such as proteins, peptides, toxins or nucleic acids and as markers to visualize cells, cell compartments or to detect biological processes.

One particularly interesting and innovative use of the luminescence method is the so-called Array technique to identify biomolecules. Array technique is understood in the field of today's bioanalytics and diagnostics as flat, glass or polymer carriers or substrates—also called biochips—onto which a number of biomolecule sensors (nucleic acids, protein or peptides) with a known structure have been applied in a grid-like arrangement. By incubating luminescence-marked proteins or nucleic acid samples that have previously been obtained, for example from tissue or cells with the biochip, a specific bond occurs at those points on its surface that have a chemico-physical structure that is complementary to the sample. Due to the luminescent marking of the sample, this results in a detectable luminescence at the point where the bond has occurred which in turn provides direct information on the chemical and physical structure of the sample. Due to the large number of sensors that have been immobilized on the chip, the Array technology is a valuable tool for testing molecular libraries in the course of drug development.

Since the quality and applicability of the chip technology depends directly on the detection sensitivity and spectral specificity of the luminescence marker, numerous developments were carried out in the past to produce new, improved luminescence systems so that the detection limits could be significantly reduced for bioassays.

The development of luminescent substances concentrates on two classes of substances. The one is luminescent molecules, the most common of these being Fluorescein, Rhodamin, Phycoerythrin, Coumarin, Cy3, Cy5, TAMRA, ROX, OREGON GREEN®, Ethidiumbromid, TEXAS RED® and Dabcyl. The other family of compounds are the semiconductor nanocrystals obtained primarily from Groups IIB and VIA, IIIA and VA or IVA of the periodic system, the most common of these being CdS, CdSe, CdTe, ZnS and ZnSe. A particular feature of these semiconductor nanocrystals is the high quantum efficiency and the fact that unlike conventional luminescence dyes they do not tend to fade. A further remarkable point that has made the compounds referred to in literature as “quantum dots” an interesting alternative to conventional fluorescent dyes, is the fact that the absorption and emissions behavior of the quantum dots depends on their size. In concrete terms this means that with smaller particle sizes the emission spectrum shifts towards the short-wave range and vice versa.

For the development of a bioassay using the luminescent effect, this opens up the additional possibility of performing different luminescence markings not only on the basis of the choice of substance but also on the particle size. This offers an ideal basis for the optical coding of biomolecules in the context of the bioarray development.

The use of fluorescent markers in bioanalytics has been described in many places in the pertinent literature.

U.S. Pat. No. 4,777,123 describes an immunoassay principle that discloses the use of receptors marked with two different fluorogens, whereby the energy transfer from the first, excited fluorogen to the second fluorogen is reduced by the presence of the ligand to be detected.

The subject of U.S. Pat. No. 5,324,633 are bioarray methods to detect fluorescence-marked receptors bonded to biopolymers with the aid of a photon counter or confocal microscopy.

Xanthene fluorescent dyes with an excitation frequency of 450-650 nm for cell detection with the aid of marked antibodies are described in U.S. Pat. No. 5,066,580.

In U.S. Pat. Nos. 3,998,943, 3,996,345, 4,174,384, 4,199,559 and 4,261,968, ligand receptor assays are disclosed whose basic principle lies in marking a receptor and/or ligand with a fluorescent dye whose emission behavior is detected depending on the ligand-receptor bond.

U.S. Pat. No. 5,319,209 discloses fluorescent sensors, amongst others, in the form of benzofuran-isophthalate that are suitable to measure ion concentrations in cells.

The production of semiconductor nanocrystals and/or quantum dots with corresponding optical characteristics is state-of-the-art and has been described variously in the pertinent literature: Kortan et al., J. Am. Chem. Soc. Vol 112, 1327, 1990; Colvin et al., Nature, Vol. 370, 354, 1994; Murray et al., J. Am. Chem. Soc. Vol. 115, 8706, 1993; Hirai et al., J. Phys. Chem. B, Vol. 103, 4228, 1999; Dabbousi et al., J. Phys. Chem. B, Vol. 101, 9463, 1997; Hines et al., J. Phys. Chem. Vol. 100, 468, 1996; Danek et al., Chem. Mater. Vol. 8, 173, 1996, Steigerwald et al., J. Am. Chem. Soc. (1988) 110: 3046-3050 and Lianos et al., Chem. Phys. Lett. Vol. 125, 299, 1986.

However, the actual synthesis alone is not decisive for the use of luminescent substances in bioanalytics. The possibility of a covalent bond between the luminescence marker and corresponding biosubstances is just as important. With luminescence dyes, functional groups are primarily introduced in the form of N-hydroxysuccinimidyl esters or isothiocyanate functions that bond with the amino groups of the biomolecule.

Quantum dots, on the other hand, are always functionalized according to two methods: On the one hand, the surfaces are converted with functional mercapto-compounds such as mercaptopropionic acid, dihydroliponic acid, thioglycolic acid, mercaptosilanes, that are then coupled to the corresponding biomolecules preferably via the carboxyl function (Gerion et al., J. Phys. Chem. Vol. 105, 8861, 2001; Mattoussi et al., J. Am. Chem. Soc. Vol. 122, 12142, 2000; Chan et al., Science Vol 281, 2016, 1998; Mitchell et al., J. Am. Chem. Soc. Vol. 121, 8122, 1999) or the quantum dots are encapsulated in a polymer matrix or dendrimers (“capping”). Lemon et al. (J. Am. Chem. Soc. Vol. 122, 12886, 2000), Sooklal et al. (Adv. Mater. Vol. 10, 1083, 1998) and Lakowicz et al. (J. Phys. Chem. Vol. 103, 7613, 1999) describe the encapsulation of the semiconductor nanocrystals with the aid of polyamidoamine dendrimers. Encapsulations using polystyrene-co-vinyl pyridine, polystyrene, silica gel or polylauryl methacrylate are described by Zhao et al. (Chem. Mater. Vol. 14, 1418, 2002), Han et al. (Nature Biotech., Vol. 19, 631, 2001), Correa-Duarte et al. (Chem. Phys. Letters, Vol. 286, 497, 1998), Chang et al. (J. Am. Chem. Soc. Vol. 116, 6739, 1994) and Lee et al. (Adv. Mater., Vol. 12, 1102, 2000).

Quantum dots encapsulated in mercapto-components and diaminocarboxyl acids as well as semiconductor nanocrystals produced in the gas phase with the aid of a spray system are the subject matter of U.S. Pat. Nos. 6,114,038 and 5,906,670.

The production of semiconductor nanocrystals from Groups III-V and II-VI that are able to couple affinity ligands is dealt with in U.S. Pat. Nos. 6,207,397, 5,251,018, 5,751,018, 5,262,357 and 5,505,928.

The subject matter of U.S. Pat. Nos. 6,326,144 and 6,207,229 are coated semiconductor nanocrystals with an affinity bond to biological components and monodisperse, light-emitting nanocrystals coated with ZnS, ZnSe, CdS or CdSe.

U.S. Pat. Nos. 5,293,050 and 5,354,707 deal with light-emitting semiconductor layers of silicon for circuits in which the second silicon layer contains at least one quantum dot.

U.S. Pat. No. 5,525,377 describes 20 to 100 Å nanoparticles of doped semiconductor compounds coated with polymethyl methacrylates for use as cathode ray tubes or electroluminescence displays.

The use of semiconductor nanocrystals in the form of ZnS, ZnSe, ZnTe, CdS, CdSe, CdTe, HgS, HgSe, HgTe, GaS, InAs, InP and InSb as electron transport media to produce electroluminescence devices that emit visible light in different wavelengths when a voltage is applied are dealt with in U.S. Pat. No. 5,537,000.

Laser-intensifying crystals doped with transition metals, consisting of Group II and VI, are the subject matter of U.S. Pat. No. 5,541,948.

A glass matrix doped with luminescent nanocrystals is described in U.S. Pat. No. 5,585,640. Temperatures over 1000° C. are needed to produce the doped, prefabricated glass forms.

U.S. Pat. No. 5,770,299 discloses semiconductor nanocrystals consisting of CdS and/or III-V components or II-VI semiconductor nanocrystals as pigments for paints.

A solid phase synthesis of fluorescence-marked peptide libraries on commercial silica gel beads is described in U.S. Pat. No. 5,789,162.

Luminescence semiconductor nanocrystal sensors for biological applications that are coated with silane derivatives and can bond affinity molecules are dealt with in U.S. Pat. No. 5,990,479.

U.S. Pat. No. 5,043,265 discloses fluorescence particles in the form of ZnS—Ag and Y₂O₂S—Eu as labels for immunoglobulins and polynucleotides.

The production of nanoparticles from transition metals, lanthanoids or actinoids in the presence of cation exchanger dispersed in an inert gel polymer in the form of nucleic acid or protein is the subject matter of U.S. Pat. No. 5,985,353. The cations bonded to the exchangers are precipitated to nanoparticles by adding corresponding anions.

Phycobiliprotein markers, that can reduce the degree of spectral overlap between the excitation and emission frequency, corresponding to a significant Stokes shift, are described in U.S. Pat. No. 4,666,862 and by Oi et al. (J. Cell. Biol. Vol. 93, 891 1982).

A further group of light-emitting substances are the so-called up-converting phosphors whose special feature is that they emit short-wave radiation against the excitation radiation. Substances of this type primarily consist of compounds of oxygen and/or sulphur and/or fluorine with the lanthanoids or yttrium.

If used in bioassays, these substances have the advantage that their absorption and emission frequencies are not disturbed by the autofluorescence of the carrier systems such as carrier particles or microtitre slides, a phenomenon that still poses problems in today's bioanalytics when using any type of carrier system.

U.S. Pat. Nos. 5,674,698 and 5,698,397 describe a series of molecular sensors in the form of doped up-converting phosphors such as Na-yttrium fluoride, lanthanum fluoride, gadolinium fluoride and yttrium oxysulphide that are used in biological and other assays by means of laser excitation.

Up-converting phosphor particles encapsulated in polyacrylate micro-carriers that are transparent for the excitation and emission frequencies and can enter into covalent bonds with protein sensors via functional groups are dealt with in U.S. Pat. No. 5,132,242.

Up-converting phosphors for the detection of nucleic acid sequences and immunoassays are described by Corstjens et al. (Clin. Chem. Vol. 47, 1885, 2001) and Niedbala et al. (Anal. Biochem. Vol. 293, 22, 2001), silane-coated up-converting phosphors by Hampl et al. (Anal. Biochem. Vol. 288, 176, 2001).

Luminescent metal porphyrins that can be used to detect biological substances are the subject matter of U.S. Pat. No. 6,004,530.

The luminescence markers known from the state of the art in the form of single molecules or nanocrystals have the disadvantage that their use, particularly in bioassays, requires complicated preparation in terms of both the time and procedure involved. Thus, synthesis or preparation times of several hours up to several days (U.S. Pat. No. 5,132,242) in conjunction with complicated procedures are required: work in a nitrogen inert gas atmosphere, distillations, work in organic media. Moreover, the products, wherever these are organic polymers, have a marked autofluorescence, cf. Han et al.

The most serious limitation of the known luminescence systems is that normally only one or very few luminescence particles (semiconductor nanocrystals or up-converting phosphor crystals) can be bonded for each biomolecule to be marked. Consequently, this limitation effectively reduces the detection sensitivity of the bioassay. The situation is similar for luminescence molecules, where only very few can be bonded per biomolecule so that it is difficult to detect a signal emitted from a marked biomolecule. In addition, coupling the luminescent substance to the biomolecule (e.g. antibody) may render the latter inactive. However, the luminescence-marked micro- or nanoparticles that have been developed as an alternative all have luminescence markers coupled to the surface of the particle that are difficult to produce, apply and detect; cf.: Fornusek and Vetvicka, “Polymeric Microspheres as Diagnostic Tools for Cell Surface Marker Tracing,” in CRC Critical Reviews in Therapeutic Drug Carrier Systems, 2, pp. 137-174, 1986. The article contains a critical overview of this technique, and other references quoted here: Kaplan et al. (Biochimica et Biophysica Acta, Vol. 728, 112, 1983) as well as U.S. Pat. Nos. 3,853,987, 4,035,316, 4,105,598, 4,108,972, 4,224,198 and 4,326,008.

### SUMMARY OF THE INVENTION

The object of the present invention is to avoid the disadvantages of the luminescence carrier systems known from the state of the art and to provide transparent, non-autofluorescent micro and submicroparticles with an adaptable porosity that contain multiplex luminescent substances. Thanks to the possibility of encapsulating a number of luminescent agents in the form of both molecules and nanoparticles or colloids, the detection sensitivity for biomolecules in the context of bioanalytics and diagnostics can surprisingly be improved to such an extent that even single molecules can be detected. Moreover, the encapsulation of several luminescence substances with varying emission behaviors allows a variety of optical codings of the biomolecule.

This task is enabled in accordance with the invention through the encapsulation of luminescent substances in a transparent matrix of silica gel that does not affect the essential luminescence properties of the basic substance.

Furthermore, this task is solved in accordance with the invention by an inverse suspension method whereby a mixture consisting of a silica gel sol and the luminescent substance are dispersed in an organic phase that is not miscible with water and then polycondensed. This produces three-dimensionally cross-linked, pearl-shaped polymer carriers that contain the encapsulated luminescence substance.

Silica sols are produced according to a method that is generally known from the state of the art through the hydrolysis of alkoxysilanes with the aid of diluted mineral acids (Dave et al. in: Immobilized Biomolecules in Analysis, editors, Cass and Ligler, Oxford University Press, 1998; WO 02/09125 A1).

Silicon orthoesters of aliphatic alcohols can be used as alkoxysilanes, preferably methyl, ethyl or propyl ester, either singly or in mixtures. The corresponding luminescence substances are added to the silica sol in the second step. In order to be able to more homogeneously mix the heterogeneous sol-luminescence substance mixture the reaction is carried out in an ultrasonic bath or using an ultrasonic sonotrode. Exposure usually lasts for half a minute. The colloid disperse mixture obtained in this way is then dispersed in an organic solvent that is not miscible with water, usually by stirring. Pearl-shaped sol drops form that then polycondense to three-dimensionally cross-linked silica gels by subsequently adding a diluted base.

The polarity characteristics of the sol and the organic dispersion phase are chosen on the one hand to produce a stable suspension and on the other so that the added luminescence substance has a high affinity to the sol phase so that there is a clear separation between the sol and organic phase and consequently the added luminescent compound is homogeneously distributed only in the sol phase.

The overall production process including preparation of the silica gel sol takes around 20 to 50 minutes depending on the formulation.

### DETAILED DESCRIPTION OF THE INVENTION

The starting point for the synthesis of the luminescence particles are silica sols that are produced according to known methods through the hydrolysis of suitable alkoxy silanes in an acidic, aqueous environment. Silanes with a C1 to C3 ester residue are preferably used for the process and product. This, together with a defined concentration of the silanes, surprisingly allows the production of completely transparent particles. The concentrations of alkoxy silanes in the initial formulation are between 40 and 90% by volume, preferably between 65 and 80% by volume. Exposing the silanes to ultrasound in the presence of an aqueous acid solution whose volume percent in the silane formulation is generally between 10 and 40% by volume and whose concentration is normally between 0.01 and 0.5 Mol/litre produces clear sols. The exposure time depending on the acid concentration is 5 to 20 minutes. The silica sols obtained in this way can be stored a number of days at deep-freeze temperatures (approx. −18° C.) or can optionally be processed immediately.

A further feature of the silica gel technology in accordance with the invention is that the porosity of the particles obtained can be varied over a wide range, a parameter that none of the other processes known from the state of the art offers in this way.

The porosity of a particle carrier medium plays a decisive role in all bioassays or separation processes inasmuch as the specific detectability of the analytes depends on their bonding to the ligands and/or receptors immobilized on the carrier. The extent and quality of this bonding process is directly affected by the accessible surface of the carrier. The latter is directly determined by the porosity of the carrier. This can surprisingly be adjusted with the aid of the products and processes in accordance with the invention by adding certain substances to the sol. These substances, generally called porogens, are added to the sol before dispersion. Preferred substances that are used as additives are those that do not affect the absorption-emission behavior of the carrier. Examples of these that do not restrict the invention include: polyethylene glycol, polyvinyl alcohol, polyacrylic acid, polyamino acids, polysaccharides, proteins or polyvinyl pyrrolidone. They are encapsulated in the spherical particles during the dispersion and cross-linking process. The volume percent of organic polymer solutions that occur as 1 to 10% aqueous solutions is between 1 and 20% relative to the sol phase.

A further parameter to adjust the porosity results from the choice of the composition of the various di-, tri- or tetrasubstituted silane components. Thus, gels of purely tetrasubstituted esters in principle have a lower porosity than sols or gels produced through the addition of di- or tri-substituted ester compounds. Selective admixture of di- or trifunctional ester compounds allows the production of pore widths in a range from 50-200 nm. The concentration of di- and trifunctional silanes is normally 1-10, preferably 1 to 5% by volume, relative to the overall silane concentration.

A further, essential aspect of the products and processes in accordance with the invention is that the particle size can be controlled by both the type and manner of mechanical stirring as well as the viscosity of the silica sol. As is known, the particle size is adjusted during suspension polymerization by the stirring speed in such a way that the particle size always drops with an increasing stirring speed. The correlation of these parameters also applies for the present invention, i.e., stirring speeds >1000 rpm generally lead to particle sizes <50 μm, those >5000 rpm to particle sizes <10 μm. In order to obtain even finer particles in the sub-micrometer range, dispersing tools are needed that work, for example, according to the rotor-stator principle and have a rotor speed of >10,000 rpm (e.g. ULTRA-TURRAX®). Treatment with ultrasound also produces particle sizes in the range from 0.5 to 10 μm.

Apart from the purely mechanical parameters, it could be shown that the viscosity of the sol also has a decisive influence over the particle size. Lowering the sol viscosity generally leads to an analogous reduction of the particle size and vice versa; increasing viscosities lead to an increase in the size of the particles. The viscosities needed to obtain the products in accordance with the invention with the desired particle sizes from 0.5 to 50 μm lie within the range of 5 to 300 cP.

After preparing the sol with the desired characteristics the corresponding luminescence marker is added in the next step and homogeneously mixed with the sol, preferably with the aid of an ultrasonic treatment. Those compounds that are able to emit photons at a frequency different from the absorption frequency after absorbing radiation of a certain wavelength and which are able to enter a homogeneous mixture with the silica gel sol can be used as luminescent substances.

A variety of substances of this type are described in the literature listed as references. Examples of luminescent molecules and markers that do not restrict the product in accordance with the invention are: Fluorescein, Rhodamin, Coumarin, Dansylchlorid, Ethidiumbromid, TEXAS RED®, Phycoerythrin, OREGON GREEN® or CASCADE BLUE® as well as the compounds known under the trade names BODIPY®, SYBR GREEN®, TOTO-1® or YOYO-1® as well as derivatives of these products. Compounds from the series II-VI such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe or HgTe as well as nanocrystals in the group III-V such as GaAs, InGaAs, InP or InAs can be used as semiconductor nanocrystals. Semiconductors from the group IVA such as germanium are also suitable.

Suitable examples of up-converting phosphors are compounds of rare earths or elements from the group IIIB such as: Na-yttrium fluoride, lanthanum fluoride, lanthanum oxysulphide, yttrium oxysulphide, yttrium fluoride, yttrium gallate, gadolinium fluoride, barium-yttrium fluorides, gadolinium oxysulphide as well as compounds of the above type doped with activator pairs such as ytterbium/erbium, ytterbium/thulium or ytterbium/holmium. Other suitable up-converting phosphors include chelate compounds of erbium, neodymium, thulium, holmium and praseodym.

Luminescent proteins such as rhodopsine, the green fluorescent protein (GFP) and metal porphyrins can also be used as luminescence substances in an analogous manner.

The concentration of luminescence substances that are added can be varied over a wide range depending on the demands made on the corresponding bioanalysis or diagnostic. Concentrations of 1-10% by weight of the marker substance are usually adequate to achieve a clear luminescence. These concentrations exceed the luminescence marker concentrations that can be used per analyte in conventional assays by several powers of ten.

The excellent miscibility of the silica sols with the various substances has surprisingly enabled magnetic compounds in the form of magnetic colloids and/or nanoparticles to be simultaneously encapsulated together with the luminescence substances. This allows a completely new combination of characteristics that permits the parallel use of the luminescence effect and magnetic separation. This opens up completely new perspectives for the development of highly efficient bioassays with which a very large number of biomolecules (“molecular library”) can be tested that were formerly unavailable to the previous arrays performed solely on polymer or glass substrates.

Magnetic substances that can be used are the known ferro-, ferri- or superparamagnetic substances as well as ferrofluids, many of which have been described in the pertinent literature: Br. Patent 1 439 031, Shinkai et al.,Bio-catalysis Vol 5, 61, 1991, Kondo et al., Appl. Microbiol. Biotechn. Vol. 41, 99, 1994.

The concentrations of magnetic colloids are all selected so that the transparency of the particles with respect to the absorption and emission behavior of the luminescence marker is not affected. They are normally between 10 and 30% by weight relative to the silica gel particles.

In the third step of the process the silica gel-luminescence marker mixtures are dispersed in an organic phase by stirring. Those solvents that are not miscible with water and allow a stable dispersion of the silica sol are suitable as dispersing agents.

Dispersing agents of this kind are generally known from the state of the art and described in WO 02/09125 as well as by Laane et al. (“Biocatalysis in Organic Media”, Laane et al. editors, Elsevier, Amsterdam, pp 65, 1987). Solvents that meet these requirements include hexane, trichloroethylene, petroleum ether, toluene, chloroform, 1,1,1-trichloroethane, carbon tetrachloride, heptane. Mixtures of the above solutions that have a density of approx. 1 g/cm³ are well suited for dispersing. The volume ratios of organic phase to hydrosol are normally 8:1 to 30:1.

With a view to a narrower distribution of particle sizes and a better dispersing behavior, one or more surfactants in the form of tensides, dispersion stabilizers or emulsifiers can be added to the organic phase. Examples of these include: Propylene oxide-ethylene oxide block copolymers, polyhydroxy fatty acid-polyethylene glycol block copolymers, polyethylene glycol-ether derivatives, sorbitan fatty acid esters, block copolymers of castor oil derivatives, polyethylene glycol-castor oil derivatives, polyoxyethylene sorbitan fatty acid esters, alkylphenyl polyethylene glycol derivatives, polyglycerol ester, modified polyester, polyoxypropylene ethylene diamine block copolymers, polyethylene glycols, polyoxyethylene derivatives, polyoxyethylene alcohol derivatives. Substances of this type are known on the market, amongst others under the trade names: RENEX®, ESTOL®, PRISORINE®,HYPERMER®, PLURONIC®, TWEEN®, EUMULGIN®, PRIPOL®, TETRONIC®, BRIJ®, LAMEFORM®, ARLACEL®, SPAN®, DEHYMULS®, SYNPERONIC® OR TRITON®. The tenside concentrations relevant for the production of the particles are between 0.1 and 15%, preferably between 0.5 and 6% by volume and/or weight.

In the last step of the process the sols are mixed with a diluted base during the dispersion process so that the former polycondenses to a solid gel. The sols are normally fixed to the desired gel particles within a few seconds (3 to 20 seconds). Accordingly, the mechanical dispersion process only takes a few seconds. The higher the selected base concentration, the shorter the gelation and parallel mechanical dispersing process. Ammonia in the form of a 1 to 12% aqueous solution is preferably used as a base. Other bases such as NaOH, diethylamine or KOH can also be used. The volume ratios of base to sol are normally between 1:2 and 1:4.

As a result of the very rapid gelation reaction, the overall particle production process including encapsulation of the luminescence markers can be carried out within one hour. This represents a time saving of up to 90% over all conventional methods to produce luminescent particles.

The production process is followed by washing several times with alcohol and water. The magnetic carriers that are obtained are normally stored in water. The silica gel particles that are obtained can be used directly for further functionalization.

In order to couple the luminescence particles to the corresponding biomolecules such as proteins, antibodies, peptides, enzymes, nucleic acids, oligosaccharides, all of which act as target substances, receptors, bioligands or analyte sensors, the generally known activation and coupling methods for silica gel and/or silanised carriers are used. These include reactions with functional alkoxy silanes that have, for example, amino, epoxy, mercapto, isothiocyanate, acrylic or halogen groups. Examples of such activation and coupling agents include: 3-aminopropyl-triethoxysilane, 3-aminopropyltrimethoxysilane, 3-glycidyl-oxypropyltrimethoxysilane, 3-glycidyloxypropylmethyl diethoxysilane, 3-mercaptopropyl trimethoxysilane, 3-isothiocyanatopropyl triethoxysilane, methacryloxypropyl triethoxysilane, chloropropyl triethoxysilane. The introduction of carboxyl, hydroxyl or aldehyde groups via a corresponding derivation of the aforementioned alkoxysilanes, e.g. with amino carboxylic acids, glutardialdehyde or hydrolysis of the epoxy groups with acids or bases to hydroxy derivatives is also generally known from the state of the art. Similarly, the reaction of the epoxy-activated carriers with carboxylic acids, sulphites, thiosulphates, amino-substituted carboxylic acids such as, for example, nitrilo-triacetic acid or imino-diacetic acid according to the known methods also leads to a metal chelate carrier. Activation of the silica gel particles through photoactive agents, for example those carrying arylazide or diazirin functions, that are initially coupled to the carrier by UV irradiation and can then react with the bioligands and/or biomolecules, is also easy to perform. Substances of this type include: N-hydroxysuccinimidyl-4-acidobenzoate, [2-nitro-4-[3-(trifluoromethyl)-3H-diazirine-3-yl]phenoxy]acetyl-N-hydroxysuccinimidate, N-hydroxysuccinimidyl-(4-acidophenyl)-1,3′-dithiopropionate, N-[m-[3-(trifluoromethyl)diazirine-3-yl]phenyl]-4-maleimidobutyramide.

A detailed description of the various activations, functionalisations and couplings is not needed at this point since the special reaction methods are generally known and can be used by an expert in this field at any time (cf. Vansant et al., in: ∂Characterization and Chemical Modification of the Silica Surface”, edited by Delmon and Yates, Elsevier, Amsterdam, 1997; “Scientific and Clinical Applications of Magnetic Carriers”, Häfeli et al. (editors), Plenum Press, New York, 1997; Shriver-Lake in: “Immobilized Biomolecules in Analysis”, Cass and Ligler, editors, Oxford University Press, 1998; WO 99/01766; Lottspeich and Zorbas editors, in: “Bioanalytik”, Spektrum Verlag, Heidelberg, 1998). The principle embodiments should thus in no way be construed as limiting disclosures.

The application of the luminescence particles in accordance with the invention covers all areas of bioanalytics and diagnostics that use color reactions, luminescence or radioactivity to detect or quantify specific substances or analytes. Examples include immunoassays in the form of radio or enzyme immunoassays, nucleic acid detection, proteom analysis, DNA sequencing, phage display, cell marking, nucleic acid array or cell marking. Moreover, the particle luminescence markers can be used in flow cytometry or a fluorescence-activated cell sorter (FACS®) or to test DNA or protein libraries.

The following examples describe the products and processes in accordance with the invention in more detail without limiting these to the examples.

### EXAMPLE 1

5 ml of tetramethoxysilane are exposed to ultrasound in an ultrasonic bath together with 2 ml of 0.05 M HCl for 10 minutes at room temperature. 2 ml of the clear sol that is obtained are mixed with 1 ml of a 0.05% Rhodamin B-solution. This mixture is then added to 25 ml of hexane containing 0.4 ml Korantin (BASF). The formulation is dispersed with a dispersing machine (Ultra-Turrax) for 3 seconds at 20,000 rpm. After adding 1 ml of a 1% ammonia solution it is dispersed for a further 5 seconds. After a further 5 minutes the particles are precipitated by means of a two-minute centrifugation. The excess is decanted off and rinsed three times with ethanol, acetone and water, approx. 10 ml in each case. Luminescence particles with a particle size of 1-3 μm are obtained.

The particles obtained are subsequently washed a number of times with anhydrous toluene and then reacted in an argon atmosphere with 5 ml of anhydrous toluene and 2 ml of 3-glycidyloxypropyl trimethoxysilane for 3 hours at 90° C. by stirring. It is then rinsed five times with toluene and acetone.

100 mg of the product obtained is then washed three times with 0.5 molar phosphate buffer, pH 8.5, and subsequently incubated with 2 ml of the same buffer, in which 10 mg of streptavidin has been dissolved, at 40° C. for 5 hours. The product is then washed again five times using a centrifugal step in each case with 0.05 M phosphate buffer, pH 7.2. In order to block any remaining epoxy groups the coupled product is stored for 24 hours in a 1% ethanolamine solution containing 0.1% serum albumin at room temperature. It is then washed a number of times with 0.05 Tris/HCl buffer, pH 8.0.

This results in a product that can be used to bond or mark biotinylated biomolecules.

### EXAMPLE 2

2 ml of the silica sol that has been produced analogous to Example 1 is mixed with 10 mg of CdS-semiconductor nanocrystals, with a mean particle size of 138 nm, that have been synthesized according to a specification from Sooklal et al. (Adv. Mater., Vol. 10, 1083, 1998), and then exposed to ultrasound for 2 min. at room temperature. The mixture is dispersed at 20,000 rpm for 5 seconds in 25 ml of toluene containing 2.5% by volume of dissolved Span 60 and 0.5% by volume of dissolved Tween 80, with the aid of an Ultra-Turrax. After adding 1 ml of a 6% ammonia solution it is then dispersed for a further 5 seconds. The particles are then separated and prepared analogous to Example 1. Luminescence particles with a mean particle size of 3.6 μm are obtained with an emission maximum of 510 nm.

In order to activate the particles, 75 mg of luminescence particles are irradiated for 20 minutes in the presence of [2-nitro-4-[3-(trifluoromethyl)-3H-diazirine-3-yl]phenoxy]acetyl-N-hydroxysuccinimidate (formulation: 5 mg dissolved in 0.5 ml ethanol-water (1:1)), with the aid of the Stratalinker UV 2400 (Stratagene). Biomolecules, ligands or receptors containing amino groups such as nucleic acids, proteins or antibodies can be coupled to the activated product after washing with ethanol and water according to the known methods (Matson and Little, J. Chromatogr., Vol. 458, 67, 1988).

### EXAMPLE 3

0.5 ml of tetraethoxysilane are mixed with 0.1 ml of water and 0.08 ml of 0.1 M HCl and exposed to ultrasound for 10 minutes at room temperature in an ultrasonic bath. 0.2 ml of the sol that is obtained are mixed with 5 mg (YYbEr)₂O₂S, which has been produced in accordance with a specification from Hampl et al. (Anal. Biochem., Vol. 288, 176, 2001), and treated for 5 minutes in an ultrasonic bath. 30 mg of magnetite powder (Bayferrox 318M, Bayer, FRG) are then added to the mixture. The mixture is exposed to ultrasound for a further 2 minutes. The mixture is then dispersed by stirring (Ultra-Turrax) at 12,000 rpm in 3 ml of trichloroethylene in which 2% by volume of Dehymuls HRE70® and 0.5% by volume of Prisorine 3700® have been dissolved. 0.08 ml of a 6% aqueous ammonia solution are added during dispersion. The mixture is stirred for a further 5 seconds. Separation and preparation of the luminescence particles obtained is analogous to Example 1.

Luminescence particles with a mean particle size of 6.7 μm are obtained.

The product is dried for 3 hours in a vacuum before being washed five times with dried toluene after respective magnetic separation and then boiled under reflux for 12 hours after adding 3 ml of toluene and 0.25 ml of 3-aminopropyltriethoxysilane. The magnetic particles are again magnetically separated and washed 3 times with approx. 5 ml of toluene and chloroform. They are then dried for several hours in a vacuum. The amino-modified product is then converted with a 6% g lutar aldehyde solution in 4 ml of a 0.1 M Na-carbonate buffer, pH 9.0, for 2 hours at 35° C. It is then thoroughly rinsed with 0.1 M phosphate buffer, pH 7.2.

Biomolecules bearing amino groups such as proteins, peptides, nucleic acids or oligonucleotides with amino groups substituted at the 5′-end, can then be coupled to the aldehyde-functionalized luminescence particles that are obtained, in accordance with the known methods. The luminescence particles that have been functionalized in this way can be used as sensors in protein or nucleic acid arrays.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. Luminescent silica gel particles containing a transparent silica gel matrix, said transparent silica gel matrix having at least one luminescent substance, said particles exhibiting at least one property selected from the group consisting of:
the size of said particles being at least 0.5 μm;
said at least one luminescent substance of said particles being selected from the group consisting of luminescent organic compounds, up-converting phosphors and luminescent proteins;
said particles containing a magnetic colloid; and
said silica gel matrix of said particles including functional groups which can be coupled with biomolecules.

2. Particles according to claim 1, wherein said particles are not self-fluorescent.

3. Particles according to claim 1, wherein said at least one luminescent substance is encapsulated in said particles.

4. Particles according to claim 1, wherein said at least one luminescent substance displays a phenomenon selected from the group consisting of fluorescence, phosphorescence, chemoluminescence, electroluminescence and luminescence energy transfer.

5. Particles according to claim 1, wherein the concentration of said at least one luminescent substance is 1 to 10%-wt.

6. Particles according to claim 1, wherein said at least one luminescent substance has an emission frequency and wherein any two of said at least one luminescent substance display different emission frequencies.

7. Particles according to claim 1, wherein said at least one luminescent substance is a molecule having an excitation frequency and an emission frequency, and wherein said excitation frequency is higher than said emission frequency.

8. Particles according to claim 1, wherein said at least one luminescent substance consists of semiconductor nanocrystals formed from elements selected from the groups of the periodic system consisting of Group IIIA, Group VA, Group IEB, Group VIA and Group IVA.

9. Particles according to claim 8, wherein said luminescent semiconductor nanocrystals are doped with at least one of copper or silver additives.

10. Particles according to claim 1, wherein said at least one luminescent substance has an excitation frequency and an emission frequency and wherein said excitation frequency is lower than said emission frequency.

11. Particles according to claim 10, wherein said at least one luminescent substance is a microcrystalline compound selected from the group consisting of rare earths and yttrium having at least one element from the Group VIA or the Group VIIA.

12. Particles according to claim 1, wherein said at least one luminescent substance is a metal-chelate compound having a central atom, said central atom being selected from the group of the periodic system consisting of Group VIII, Group IB, Group IIB and rare earths.

13. Particles according to claim 1, wherein said at least one luminescent substance is a pyrrole dye.

14. Particles according to claim 1, wherein said luminescent substance is a luminescent protein.

15. Luminescent polymer particles comprising:
a transparent silica gel matrix;
at least one luminescent substance in said transparent silica gel matrix; and
a magnetic colloid.

16. Particles according to claim 15, wherein said magnetic colloid is selected from the group comprising ferro-magnetic compounds, ferri-magnetic compounds, superparamagnetic compounds and ferrofluids.

17. Particles according to claim 15, wherein said magnetic colloid is present in a concentration of 10-50% by weight relative to the polymer particle.

18. Particles according to claim 15, wherein said silica gels have functional groups that can be coupled to at least one biomolecule, said at least one biomolecule being selected from the group consisting of proteins, peptides, cell receptors, nucleic acids, nucleic acid fragments, polysaccharides, oligosaccharides, antibodies, antibody-fragments, streptavidin, avidin, biotin and enzymes.

19. A process for the production of luminescent silica gel particles which contain a transparent silica gel matrix having at least one luminescent substance in said silica gel particles, said process comprising the steps of:
condensing a mixture consisting of a diluted acid and alkoxysilanes to a clear silica sol;
homogenously mixing the clear silica sol with at least one luminescent substance to form a sol-luminescence substance mixture;
dispersing the sol-luminescence substance mixture in an organic phase that is not miscible with water; and
adding a base to the sol-luminescence substance mixture during or after said dispersing step in order to cross-link said sol-luminescence substance mixture.

20. (canceled)

21. The process according to claim 19, wherein said organic phase that is not miscible with water contains at least one surfactive substance in a concentration of 0.1 to 15% by volume

22. The process according to claim 19, wherein the volume ratio of sol to organic phase is 1:5 to 1:30.

23. The process according to claim 19, wherein said dispersing and cross-linking steps have a duration of 2 to 30 seconds.

24. (canceled)

25. A sensor for array technology or for nucleic acid sequencing comprising luminescent silica gel particles containing a transparent silica gel matrix and at least one luminescent substance in said transparent silica gel matrix, for at least one of the analysis or diagnostic testing of nucleic acids, nucleic acid fragments, proteins, peptides, antibodies, antibody fragments, cells, cell receptors and biotinylated biomolecules and testing protein or nucleic acid libraries.

26. The process according to claim 19 and further comprising the step of adding a substance selected from the group consisting of a ferro-magnetic substance, a ferri-magnetic substance and a superparamagnetic substance to the sol-luminescence substance mixture in an amount of 10-50% by weight.

27. The process according to claim 19, and further including the step of mixing an aqueous solution of organic polymer, a polysaccharide or a protein in an amount of 1-20% by volume with the sol before said dispersing step.

28. Particles according to claim 15, wherein said silica gels have functional groups that are coupled to at least one biomolecule, said at least one biomolecule being selected from the group consisting of proteins, peptides, cell receptors, nucleic acids, nucleic acid fragments, polysaccharides, oligosaccharides, antibodies, antibody-fragments, streptavidin, avidin, biotin and enzymes.
